(19) United States
(12) Patent Application Publication
(10) Pub. No.: US 2022/0087275 A1
(43) Pub. Date: Mar. 24, 2022

(54) MAGNESIUM ION AS ANTIBACTERIAL AGENT

(71) Applicants: THE STATE OF ISRAEL, MINISTRY OF AGRICULTURE & RURAL DEVELOPMENT, AGRICULTURAL, Rishon Lezion (IL); YISSUM RESEARCH DEVELOPEENT COMPANY OF THE HEBREW UNIVERSISTY OF JERUSALEM LTD, Jerusalem (IL)

(72) Inventors: Moshe SHEMESH, Modiin (IL); Doron STEINBERG, Jerusalem (IL); Ram REIFEN, Rehovot (IL)

(21) Appl. No.: 17/540,863

(22) Filed: Dec. 2, 2021

Related U.S. Application Data
(63) Continuation of application No. 16/310,436, filed on Dec. 16, 2018.
(60) Provisional application No. 62/360,496, filed on Jul. 11, 2016.

(51) Int. Cl. A23C 9/13; A23L 33/16; A23C 7/02; A23C 19/05; A23C 9/152; A61L 2/00

(52) U.S. Cl. CPC A23C 9/1322; A23L 33/16; A23C 7/02; A23C 19/054; A23V 2250/161; A61L 2/00; A23V 2002/00; A23V 2200/10; A23C 9/1522

(57) ABSTRACT

Compositions comprising magnesium enriched liquids re provided. Specifically, compositions comprising magnesium enriched milk and/or milk product, wherein a concentration of magnesium ions in said milk and/or milk product ranges from 8 mM to 25 mM, and methods of producing the same, are provided.

### CROSS-REFERENCE TO RELATED APPLICATIONS

This application is a Continuation of U.S. patent application Ser. No. 16/310,436, filed on Dec. 16, 2018, which is a National Phase of PCT Patent Application No. PCT/IL2016/051000 having International filing date of Sep. 8, 2016, which claims the benefit of priority of U.S. Provisional Patent Application No. 62/339,977, filed May 23, 2016, and U.S. Provisional Patent Application No. 62/360,496, filed Jul. 11, 2016 and entitled “MAGNESIUM ION AS ANTIBACTERIAL AGENT”. The contents of the above applications are all which are incorporated herein by reference as if fully set forth herein in their entirety.

### FIELD OF INVENTION

The present invention is directed to, inter alia, magnesium enriched products and use thereof such as for preventing biofilm formation and manufacturing cheese.

### BACKGROUND OF THE INVENTION

Despite advances in food preservation techniques, bacterial spoilage remains a leading cause of global food loss. Nearly one-third of all food produced worldwide is estimated to be lost postharvest, and much of this loss can be attributed to microbial spoilage. Dairy products constitute one of the leading sectors impacted by food loss, as nearly 20% of conventionally pasteurized fluid milk is discarded prior to consumption each year. Bacterial contamination can adversely affect the quality, functionality and safety of milk and its derivatives. It appears that the major source of the contamination of dairy products is often associated with biofilms on the surfaces of milk processing equipment. Biofilms are highly structured multicellular communities, which allow bacteria to survive in a hostile environment.

Bovine milk is highly nutritious and this makes it an ideal medium for the growth of microorganisms. It contains abundant water and nutrients (such as lactose, proteins and lipids) and has a nearly neutral pH. Since microorganisms in milk may hold spoilage and/or health risks, milk manufacturing is subject to extremely stringent regulations. These regulations include pasteurization at high temperatures, which kills most bacteria, and milk storage at low temperatures, which limits the growth of many bacteria. In addition, dairy farm pipelines are regularly cleaned with alkaline and acidic liquids at high temperatures in a cleaning-in-place (CIP) procedure. Despite these stringent conditions, some bacteria are able to overcome these obstacles. For instance, thermophilic and spore-forming bacteria are able to survive pasteurization procedures, and psychrotropic bacteria thrive at the low temperatures in which milk is stored. Moreover, bacterial spores can survive treatment with reagents commonly used in CIP procedures. Some of these bacteria produce enzymes (proteases and lipases), resulting in off-flavors and curdling in the final product.

Members of the Bacillus genus are of the most common bacteria found in dairy farms and processing plants. Moreover, they are the predominant type of Gram-positive bacteria isolated from both raw milk and pasteurized milk. Thermophilic, mesophilic and psychrotrophic strains of Bacillus have all been identified in dairy farms and/or milk. B. cereus forms abundant biofilms on stainless steel, commonly used in food processing plants and contributes to biofouling of processed food. Notably, in a commercial dairy plant B. cereus was found to account for more than 12% of the biofilms constitutive microflora. As Bacillus species are ubiquitously present in nature, they easily spread through food production systems, and contamination with these species is almost inevitable. Moreover, B. cereus spores are both highly resistant to a variety of stresses and very hydrophobic, these features allow them to adhere easily to food processing equipment. The biofilm formed by thermo-resistant Bacillus species in a milk line can rapidly grow to such an extent that the passing milk is contaminated with cells released from the biofilm. Thus, biofilms formed by Bacillus species is the major type of hygiene problems in dairy industry.

### SUMMARY OF THE INVENTION

According to another aspect, the invention provides a method for reducing or inhibiting biofilm formation within a liquid and/or improving pasteurization effectiveness of the liquid, the method comprising the step of adding a magnesium ions source to a liquid to reach a final concentration of said magnesium ions in said liquid ranging from 8 mM to 150 mM, thereby producing magnesium enriched liquid. In some embodiments, the method further comprises the step of pasteurizing the magnesium enriched liquid.

According to another aspect, the invention provides a method for treating, preventing, inhibiting, and/or reducing biofilm formation and/or reducing or breaking-down existing biofilms on a surface, the method comprises the steps of: providing a composition comprising an effective concentration of magnesium ions; and contacting said surface with said composition. In some embodiments, the effective concentration of magnesium ions is at least 20 mM.

According to another aspect, the invention provides a composition comprising a magnesium enriched milk, wherein a concentration of magnesium ions in said magnesium enriched milk ranges from 8 millimol per liter (mM) to 25 mM. In some embodiments, the concentration of said magnesium ions in said magnesium enriched milk ranges from 10 mM to 15 mM.

In some embodiments, the composition has reduced biofilm formation.

In some embodiments, the magnesium enriched milk is a pasteurized magnesium enriched milk. In some embodiments, the pasteurized magnesium enriched milk is characterized by less than 1 colony forming unit (CFU)/milliliter.

In some embodiments, the magnesium enriched milk has reduced rennet clotting time (RCT) compared to non-magnesium enriched milk (such as obtained from the same mammal or processed throughout similar processes without Mg addition). In some embodiments, magnesium enriched milk has increased curd firmness (CF, min) compared to a magnesium non-enriched milk obtained from the same mammal.

In some embodiments, there is provided an article comprising the composition of the invention. In some embodiments, the article is selected from the group consisting of a food package, a milk production and/or processing device.

According to another aspect, the invention provides a method comprising the step of adding a magnesium ions source to milk to reach a final concentration of said magnesium ions in said milk ranging from 8 mM to 25 mM, thereby producing magnesium enriched milk. In some embodiments, the method further comprises the step of pasteurizing the magnesium enriched milk.

In some embodiments, the method is for reducing or inhibiting biofilm formation in said milk. In some embodiments, the method is for improving pasteurization effectiveness. In some embodiments, the method is for increasing protein incorporation into a milk product.

In some embodiments, the magnesium ions source is a magnesium salt. In some embodiments, the magnesium salt is selected from: magnesium chloride, magnesium fluoride, magnesium sulfate, magnesium nitrate, magnesium acetate, magnesium carbonate, magnesium citrate, magnesium phosphate, and hydrates thereof.

In some embodiments, the biofilm is formed of a bacteria selected from Gram positive bacteria and Gram negative bacteria. In some embodiments, the biofilm is formed of a spore forming bacteria. In some embodiments, the bacteria are selected from the genera consisting of: bacillus, geobacillus, anoxybasillus, and pseudomona. In some embodiments, the bacteria are selected from the bacteria strains: Bacillus cereus, Bacillus subtilis, Geobacillus stearothermophilus, Anoxybacillus flavithermus, and Pseudomonas aeruginosa.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1A shows photographs of pellicle formation and colony formation of B. subtilis (NCIB3610) in the presence of 5 mM, 10 mM, 25 mM, 50 mM, 100 mM MgCl₂ or a control (with no addition of MgCl₂);

FIG. 1B is a graph demonstrating growth curves of B. subtilis NCIB3610 grown in either LBGM medium (control) or LBGM medium supplemented with 5 mM, 10 mM, 25 mM, 50 mM or 100 mM MgCl₂;

FIG. 1C shows CLSM images of fluorescently tagged B. subtilis cells (YC161 with Pspank-gfp) following 24 hours of incubation in biofilm promoting medium in the presence of with 5 mM, 10 mM, 25 mM, 50 mM or 100 mM MgCl₂;

FIG. 1D shows photographs of colony formation of B. subtilis (NCIB3610) on a solid LBGM mediums solidified with 1.5% agar that were pre-treated by spreading a solution of 5 Mm MgCl₂, 20 mM MgCl₂, 50 mM MgCl₂ or a control (with no addition of MgCl₂);

FIG. 1E shows photographs of biofilm formation by B. subtilis (NCIB3610) within orange juice enriched medium un-supplemented (LB+ Orange juice), supplemented with 50 mM MgCl₂ (LB+ Orange juice+50 mM MgCl₂) or 80 mM MgCl₂ (LB+ Orange juice+50 mM MgCl₂);

FIGS. 2A-C are bar graphs showing the effect of Mg′ ions (A), Ca′ ions (B), and Na⁺ ions (C) on transcription of the operons responsible for the matrix production (epsA-O and tapA operons);

FIG. 3A shows CLSM images of fluorescently tagged B. subtilis cells following 5 hours of incubation within milk in the presence of additional MgCl₂ concentrations as indicated;

FIG. 3B is a graph demonstrating growth curves of B. subtilis within milk to which different concentrations of MgCl₂ were added;

FIG. 4 shows CLSM images demonstrating the expression of tapA operon of B. subtilis cells grown within milk, or milk supplemented with 1 mM, 3 mM, or 5 mM MgCl₂;

FIG. 5 is a bar graph demonstrating the effect of increased concentration of magnesium ions or calcium ions on the survival of B. subtilis grown within milk, following pasteurization;

FIG. 6 is a graph showing analyses performed by Optigraph instrument (Ysebaert, Frepillon, France) of samples supplemented by either 5 mM MgCl₂ (cuvettes 7 and 8), 3 mM MgCl₂ (cuvettes 5 and 6) or 3 mM CaCl2 (cuvettes 3 and 4) in comparison to the control sample un-supplemented milk (cuvettes 1 and 2);

FIG. 7 shows soft cheese samples prepared from milk supplemented with either CaCl₂ or MgCl₂ in comparison to un-supplemented milk, the indicated concentrations represents the increase in concentration within the milk;

FIGS. 8A-B are bar graphs showing measured time until curding of the cheese begins (8A) and the curd firmness (8B) of un-supplemented milk and milk samples supplemented with 1 mM, 3 mM, 5 mM, 7 mM, 10 Mm, 15 mM or 20 Mm MgCl₂;

FIG. 9 is a bar graph showing percentage of protein in cheese produced from milk (control) and cheese produced from magnesium enriched milk having 5 mM increase in magnesium ion concentration (5 mM MgCl₂); and

FIG. 10 shows photographs of vessels of milk or milk supplemented with 3 mM, 5 mM, 10 Mm, 50 mM or 100 mM MgCl₂ containing B. subtilis cultures.

### DETAILED DESCRIPTION OF THE INVENTION

The present invention relates to magnesium enriched products. According to some embodiments, the magnesium enriched products show reduced biofilm formation and enhances the break-down of existing biofilms on a surface. According to some embodiments aspects, the magnesium enriched liquids are less susceptive to biofilm formation. In some embodiments, the liquids are intended for mammalian (e.g., human) consumption.

The present invention further relates to magnesium enriched milk formulations and magnesium enriched milk products. Surprisingly, substantial reduction of biofilm formation within milk products is achieved by means of magnesium enrichment. In some embodiments, biofilm formation within the composition of the invention is reduced, compared to milk or milk product that were not supplemented with magnesium ions. In some embodiments, suppression of bacterial formation in the composition of the invention by heat (e.g., pasteurization) is more efficient compared to milk or milk products that were not supplemented with magnesium ions.

The invention further provides a method for enriching a milk product with magnesium, the method comprises the step of adding magnesium ions to the milk product, thereby providing a magnesium enriched milk product. The invention further provides a method for inhibiting and/or reducing biofilm formation in a milk product by adding magnesium ions to the milk product so as to obtain a magnesium enriched milk product, thereby reducing biofilm formation in said milk or said milk product.

The invention is based, in part, on the surprising finding that magnesium ions inhibit biofilm formation of Bacillus species. The invention is also based, in part, on the finding that bacterial cells, in the presence of Mg²⁺, were found to exhibit increased sensitivity to heat pasteurization undertaken during milk processing. The invention is further based, in part, on the finding that incorporation of milk proteins into the curd during cheese making is improved in the presence of magnesium ions.

As demonstrated herein bellow, milk supplemented with additional 3 mM, 5 mM and 10 mM magnesium ions to its final concentration is characterized by reduced biofilm formation (see, example 3), improved pasteurization effectiveness (see, example 5), reduced rennet clotting time (RCT) (see, example 6) during cheese making, increased curd firmness of cheese (see, example 6), and higher incorporation of proteins into the cheese (see, example 7), compared to un-supplemented milk.

Without limiting the invention to any theory or mechanism of action, it is further demonstrated that the inhibition of biofilm formation may be partially attributed to an inhibiting effect of magnesium ions over extra cellular matrix formation (see, examples 2 and 4).

Compositions

According to some aspects, the present invention provides compositions comprising a magnesium enriched liquid, wherein a concentration of the magnesium ions in the magnesium enriched liquid ranges from 8 millimol per liter (mM) to 150 mM.

In some embodiments, the liquids are beverages and/or beverages products. In some embodiments, the liquids are non-dairy beverages and/or non-dairy beverages products. As used herein the term “non-dairy” include all types of products that contain no milk or milk products from a mammalian source. As used herein the term “beverage” refers to a substantially aqueous drinkable composition suitable for human consumption. Non-limiting examples of beverages include water, soft drinks, juice based on fruit extracts, juice based on vegetable extracts, plant milk (e.g., soy milk, almond milk, rice milk, coconut milk etc.), coffee, tea, and any combination thereof.

Non-limiting examples of fruit extracts include extracts from mango, pomegranate, passion fruit, berries, watermelon, strawberry, plum, pear, grape, guava, grapefruit, lemon, tangerine, papaya, pineapple, apple, cranberry, banana, orange or any combinations thereof.

Non-limiting examples of vegetable extracts include extracts from carrot, tomato, beetroot or any combinations thereof. The extracts can be in the form of juices, pulps or any combinations thereof, which goes into making of the beverages.

As used herein the terms “magnesium enriched” refers to a liquid or a product thereof (e.g., beverage, non-dairy beverage) supplemented with magnesium ions, resulting in higher concentration of magnesium ions compared to a natural concentration of magnesium in the liquid.

According to some aspects, the present invention provides compositions comprising a liquid, wherein the magnesium enriched liquid is supplemented with an additional concentration of magnesium ranging from 5 millimol per liter (mM) to 150 mM.

According to some aspects, the present invention provides compositions comprising a non-dairy beverage and/or a non-dairy beverage product, wherein the magnesium enriched non-dairy beverage and/or a non-dairy beverage product is supplemented with an additional concentration of magnesium ions ranging from 20 mM to 150 mM, 25 mM to 150 mM, 30 mM to 150 mM, 35 mM to 150 mM, 40 mM to 150 mM, 45 mM to 150 mM, 50 mM to 150 mM, 60 mM to 150 mM, 70 mM to 150 mM, 20 mM to 100 mM, 20 mM to 100 mM, 25 mM to 100 mM, 30 mM to 100 mM, 35 mM to 100 mM, 40 mM to 100 mM, 45 mM to 100 mM, 50 mM to 100 mM, 60 mM to 100 mM, 70 mM to 100 mM, 20 mM to 90 mM, 25 mM to 90 mM, 30 mM to 90 mM, 35 mM to 90 mM, 40 mM to 90 mM, 45 mM to 90 mM, 50 mM to 90 mM, 60 mM to 90 mM, 70 mM to 90 mM, 20 mM to 80 mM, 25 mM to 80 mM, 30 mM to 80 mM, 35 mM to 80 mM, 40 mM to 80 mM, 45 mM to 80 mM, 50 mM to 80 mM, 60 mM to 80 mM, 70 mM to 80 mM.

Enriched Milk Compositions

According to some aspects, the present invention provides compositions comprising a magnesium enriched milk product, wherein a concentration of the magnesium ions in the magnesium enriched milk product ranges from 8 mM to 30 mM.

As used herein, the term ‘milk’ refers to any normal secretion obtained from the mammary glands of mammals, such as human's, cow's, goat's, horse's, camel's, pig's, buffalo's or sheep's milk, and includes milk, whey, combinations of milk and whey as such or as a concentrate, and the various milk products produced therefrom. Milk typically comprises whey proteins and caseins. The ratio between whey proteins and caseins may differ between different species. For example, the protein content of cow's milk includes 20% whey proteins and 80% caseins, whereas the protein content of human's milk includes 60% whey proteins and 40% caseins.

As used herein the term “whey proteins” refers to a mixture of globular proteins. There are many whey proteins in milk and the specific set of whey proteins found in mammary secretions varies with the species, as well as other factors. The major whey proteins in cow's milk are ß-lactoglobulin and a-lactalbumin. As used herein, the term “casein” refers to α_{S1}-casein, α_{S1}-casein, β-casein, κ-casein or the combination thereof as present in milk of mammals, the different caseins are distinct molecules but are similar in structure. The different caseins are found in milk as a suspension of particles, i.e., casein micelles. The term “casein”, as used herein, further encompasses acid casein, rennet casein, hydrolyzed casein, sodium caseinate, potassium caseinate, magnesium caseinate, calcium caseinate, and combinations thereof.

In some embodiments, the mammal is selected from the group consisting of: sheep, cow, goat, camel, buffalo, pig, and a horse. In some embodiments, the mammal is a cow. In some embodiments, the milk is a cow's milk. In some embodiments, the milk is a human's milk.

The milk may be supplemented with ingredients generally used in the preparation of milk products, such as fat, protein or sugar-fractions, or the like. The milk thus includes, for example, full-fat milk, low-fat milk, skim milk, delectated milk, cream, ultrafiltered milk, diafiltered milk, micro-filtered milk, milk recombined from milk powder, condensed milk, powder milk organic milk or a combination or dilution of any of these.

As used herein the term “milk product” refers to a product derived from any processing of milk. The term “milk product” further encompasses fermented milk products. Non-limiting examples of fermented milk products include: yoghurt, kefir, curd cheese, curd, buttermilk, butter, fresh cheese and semi-solid cheese. In some embodiments, the milk product is cheese.

The term “cheese” as used herein refers broadly to all types of cheeses including, for example, cheeses as defined under the CODEX general Standard for Cheese and as defined under various state and national regulatory bodies. Exemplary classes of cheeses include, but are not limited to, firm/semi-hard cheeses, soft cheeses, analog cheeses, blended cheeses, and pasta filata cheeses, among other types of cheeses. The term “firm/semi-hard cheese” includes cheeses having a percentage moisture on a fat-free basis (MFFB) of between 54% and 69%. Examples of firm/semi-hard cheeses include Colby, Havarti, Monterey Jack, Gorgonzola, Gouda, Cheshire, and Munster, low-moisture Mozzarella, and part-skim Mozzarella, among others. The term “soft cheese” includes cheeses having a MFFB of greater than 67%. Examples of soft cheeses include standard Mozzarella, among others.

As used herein the terms “magnesium enriched milk” and “magnesium enriched milk product” refer to milk and/or milk product supplemented with magnesium ions, resulting in higher concentration of magnesium ions compared to a natural concentration of magnesium in milk and/or milk products obtained from the same mammalian source (e.g., cow). In some embodiments, a concentration of magnesium in the magnesium enriched milk and/or milk product is no more than 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 Mm, 17 mM, 18 mM, 19 mM or 20 mM higher than in milk and/or milk product obtained from the same source that were not supplemented with magnesium ions. In some embodiments, a concentration of magnesium in the magnesium enriched milk and/or milk product is at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 Mm, 17 mM, 18 mM, 19 mM or 20 mM higher than in milk and/or milk product obtained from the same source that were not supplemented with magnesium ions. In some embodiments, a concentration of magnesium in the magnesium enriched milk and/or milk product is at least 1 mM, 2 mM, 3 mM, 4 mM, 5 mM, 6 mM, 7 mM, 8 mM, 9 mM, 10 mM, 11 mM, 12 mM, 13 mM, 14 mM, or 15 mM higher than in milk and/or milk product obtained from the same source that were not supplemented with magnesium ions. Each possibility represents a separate embodiment of the present invention. In some embodiments, a concentration of magnesium in the magnesium enriched milk and/or milk product is at least 1 mM higher than in milk and/or milk product obtained from the same source that were not supplemented with magnesium ions. In some embodiments, a concentration of magnesium in the magnesium enriched milk and/or milk product is between 1 mM and 10 mM, 1 mM and 15 mM, 1 mM and 16 mM, 1 mM and 17 mM, 1 mM and 18 mM, 1 mM and 19 mM, or 1 mM and 20 mM higher than in milk and/or milk product obtained from the same source that were not supplemented with magnesium ions. Each possibility represents a separate embodiment of the present invention.

In some embodiments, the concentration of magnesium ions in the enriched milk or milk product ranges from 7 mM to 30 mM, 7 mM to 25 mM, 7 mM to 20 mM, 7 mM to 19 mM, 7 mM to 18 mM, 7 mM to 16 mM, 7 mM to 15 mM, 7 mM to 14 mM, 7 mM to 13 mM, 7 mM to 12 mM, 7 mM to 11 mM, 7 mM to 10 mM, 8 mM to 30 mM, 8 mM to 25 mM, 8 mM to 20 mM, 8 mM to 19 mM, 8 Mm to 18 mM, 8 Mm to 16 mM, 8 mM to 15 mM, 8 mM to 14 mM, 8 Mm to 13 mM, 8 Mm to 12 mM, 8 Mm to 11 mM, 8 mM to 10 mM, 10 mM to 30 mM, 10 mM to 25 mM, 10 mM to 20 mM, 10 mM to 19 mM, or 10 mM to 18 mM, 10 mM to 17 mM, or 10 mM to 16 mM, 10 mM to 15 mM, 10 mM to 14 mM, 10 mM to 13 mM 10 mM to 12 mM, or 10 mM to 11 mM. Each possibility represents a separate embodiment of the present invention.

In some embodiments, biofilm formation within the magnesium enriched milk and/or milk product is inhibited. In some embodiments, the composition of the invention is characterized by reduced biofilm formation compared to milk or milk products that were not supplemented with magnesium ions. In some embodiments, biofilm formation within the composition of the invention is reduced compared to that within milk and milk products from the same origin that were not supplemented with magnesium ions. In some embodiments, there is at least 10%, 20%, 30%, 40%, 50%, 60%, 90%, or 100% reduction in biofilm formation within the enriched milk and/or milk product compared to that within milk and/or milk product from the same origin that were not supplemented with magnesium ions.

In some embodiments, the composition of the invention is pasteurized. In some embodiments, upon pasteurization an elimination of bacteria is achieved. In some embodiments, the pasteurized magnesium enriched milk is characterized by less than 1 colony forming unit (CFU)/milliliter. In some embodiments, the pasteurized magnesium enriched milk is characterized by less than 10 colony forming units (CFU)/milliliter. In some embodiments, the pasteurized magnesium enriched milk is characterized by less than 100 colony forming units (CFU)/milliliter. In some embodiments, the pasteurized magnesium enriched milk is characterized by less than 1000 colony forming units (CFU)/milliliter. In some embodiments, when pasteurized the composition of the invention exhibits reduction of bacterial cell's viability compared to that achieved upon pasteurization of milk and/or milk product from the same origin that were not supplemented with magnesium ions. In said embodiments, the composition of the reduction in bacterial cell's viability is at least 10%, 20%. 30%, 40%, 50%, 60%, 70%, 80%, 90%, 100% higher within the composition of the invention, compared to milk and/or milk product from the same origin that were not supplemented with magnesium ions.

In some embodiments, the composition enables more efficient pasteurization of bacteria compared to milk and/or milk product from the same origin that were not supplemented with magnesium ions. The term “pasteurization” refers in this context to heating of the substance to be treated (such as milk) typically at a temperature between 72 and 95° C. for 20 to 60 seconds, for instance at least at a temperature of 72° C. for 15 seconds.

Milk Coagulation Processes for Making Cheese

In some embodiments, the magnesium enriched milk of the invention, is processed to produce magnesium enriched cheese. Cheese typically consists of proteins and fat from milk, such as the milk of cows, buffalo, goat, or sheep. A skilled artisan will appreciate that the magnesium enriched cheese of the invention may be obtained by any suitable process known in the art, such as, e.g., by enzymatic coagulation of the cheese milk with rennet, or by acidic coagulation of the cheese milk with food grade acid or acid produced by lactic acid bacteria growth. Specifically, cheese is produced by coagulation of casein. During the process of clotting, coagulation enzymes (e.g., milk-clotting proteases) act on the soluble portion of the caseins, casein, thus originating an unstable micellar state that results in clot formation.

In one embodiment, the enriched milk of the invention is used for manufacturing cheese by rennet coagulation. In one embodiment, the enriched milk product of the invention is rennet-curd cheese. Rennet is commercially available, e.g. as Naturen® (animal rennet), Chy-max® (fermentation produced chymosin), Microlant® (Microbial coagulant produced by fermentation), all from Chr-Hansen A/S, Denmark). As used herein “chymosin” refers to an aspartic protease that specifically hydrolyzes the peptide bond in Phe105-Met106 of K-casein.

In general, milk coagulation properties may be defined as the feature of the milk to react with a clotting enzyme and form a curd with a suitable firmness in a reasonable time.

In embodiments wherein the enriched milk is processed, the rennet clotting time (RCT) ranges from 5 minutes to 18 minutes, 5 minutes to 15 minutes, 5 minutes to 14 minutes, 7 minutes to 18 minutes, or 7 minutes to 15 minutes. Each possibility represents a separate embodiment of the present invention. In some embodiments, the enriched milk of the invention is characterized by a reduced RCT compared to that of milk that was not supplemented with magnesium. In some embodiments, the RCT of the magnesium enriched milk is at least 2 minutes, 3 minutes, 5 minutes, 6 minutes, 7 minutes, 8 minutes, 9 minutes, or 10 minutes shorter compared to that of milk that was not supplemented with magnesium. In some embodiments, the RCT of the magnesium enriched milk is decreased by at least 25%, 30%, 35%, 40%, 45%, 50%, 55%, 60%, 65%, 70%, 75% compared to the RCT of milk that was not supplemented with magnesium.

As used herein, “Rennet Clotting Time (RCT)” refers to the time between the addition of a clotting enzyme and the beginning of the coagulation/clotting process.

In embodiments wherein the magnesium enriched milk product is cheese, the enriched cheese is characterized by increased curd firmness compared to that of cheese that was not supplemented with magnesium ions and/or obtained by processing milk that was not supplemented with magnesium ions. In some embodiments, the enriched cheese is a product of milk enriched by between 1 mM MgCl₂ and 15 mM MgCl₂.

In some embodiments, the increase is at least 10% increase, 15% increase, 20% increase, 25% increase, 30% increase, 35% increase, 40% increase, 45% increase, 50% increase, 55% increase, 60% increase, 70% increase, or 75% increase in curd firmness. In some embodiments, the increase is at least 1 volt increase, 2 volts increase, 3 volts increase, 4 volts increase, 5 volts increase, 6 volts increase, 7 volts increase, 8 volts increase, 9 volts increase, or 10 volts increase in curd firmness. In some embodiments, the magnesium enriched cheese is characterized by curd firmness of 9 volts to 25 volts, 9 volts to 20 volts, 9 volts to 18 volts, 9 volts to 15 volts, 10 volts to 25 volts, 10 volts to 20 volts, 10 volts to 18 volts, 10 volts to 15 volts, 11 volts to 25 volts, 11 volts to 20 volts, 11 volts to 18 volts, or 11 volts to 15 volts. Each possibility represents a separate embodiment of the present invention.

As used herein, Curd firmness refers to a measured curd firmness at a specific time point (e.g., 30 minutes) following the addition of a clotting enzyme. In some embodiments, the curd firmness is measured 30 minutes following addition of the rennet to the milk. In some embodiments, the curd firmness is measured 90 minutes following addition of the rennet to the milk. Alternatively, for comparing measured curd firmness of milk and milk supplemented with MgCl₂, other time point following the addition of the rennet to the milk may be selected.

In some embodiments, the magnesium enriched cheese is characterized by improved organoleptic properties. The term “organoleptic” as used in the present invention refers to any sensory property of a product, involving taste, color, odor, texture and mouth feel. In some embodiments, the magnesium enriched cheese is characterized by improved texture.

Methods

According to some aspects, the invention provides a method comprising the steps of adding an effective amount of magnesium ions to a liquid (e.g., beverage), thereby producing magnesium enriched liquid. A person with skill in the art will appreciate that the effective amount may differ between different types of liquids and selected applications.

In some embodiments, the method further comprises a step of pasteurizing said magnesium enriched liquid.

According to some aspects, the invention provides a method comprising the steps of adding an effective amount of magnesium ions to a non-dairy beverage or a non-dairy beverage product, thereby producing magnesium enriched non-dairy beverage and/or magnesium enriched non-dairy beverage product.

According to some aspects, the invention provides a method comprising the steps of adding an effective amount of magnesium ions to a milk or a milk product, thereby producing magnesium enriched milk and/or magnesium enriched milk product.

In some embodiments, the method further comprises a step of pasteurizing said magnesium enriched milk.

In some embodiments, the method is for reducing and/or inhibiting biofilm formation in a liquid (e.g., beverage, milk) or a milk product. In some embodiments, the method is for improving milk pasteurization. In some embodiments, the method is for increasing protein level in cheese, which is facilitated by magnesium dependent incorporation of milk proteins into the curd during cheese making.

In some embodiments, the effective amount is such that a final concentration of magnesium ions in the magnesium enriched liquid ranges from 5 mM to 100 mM. In some embodiments, the magnesium concentration in the resultant enriched liquid ranges from 5 mM to 10 mM, 5 mM to 50 mM, 5 mM to 25 mM, 5 mM to 100 mM, 5 mM to 200 mM, 5 mM to 500 mM, 10 mM to 50 mM, 10 mM to 100 mM, 10 mM to 200 mM, 10 mM to 500 mM, 50 mM to 100 mM, 50 mM to 200 mM, 50 mM to 500 mM, 100 mM to 200 mM, 100 mM to 300 mM, 100 mM to 500 mM, or 100 mM to 1000 mM. Each possibility represents a separate embodiment of the present invention.

In some embodiments, the effective amount is such that a final concentration of magnesium ions in the magnesium enriched milk and/or milk product ranges from 7 mM to 30 mM. In some embodiments, the magnesium concentration in the resultant enriched milk or milk product ranges from 7 mM to 30 mM, 7 mM to 25 mM, 7 mM to 20 mM, 7 mM to 15 mM, 7 mM to 10 mM, 8 Mm to 30 mM, 8 Mm to 25 mM, 8 Mm to 20 mM, 8 mM to 15 mM, 8 mM to 10 mM, 10 mM to 30 mM, 10 Mm to 25 mM, 10 mM to 20 mM, or 10 mM to 15 mM. Each possibility represents a separate embodiment of the present invention.

In some embodiments, the effective amount is such that a final concentration of magnesium ions in the magnesium enriched milk and/or milk product is elevated by at least 1 mM compared to that of a milk and/or a milk product obtained from the same source that was not supplemented with magnesium ions.

In some embodiments, the method comprises the step of adding a magnesium ions source to the milk to achieve a final concentration of magnesium ions in the milk ranging from 8 mM to 25 mM, thereby producing magnesium enriched milk.

In some embodiments, the magnesium ions source is an aqueous solution of magnesium hydroxide or a magnesium salt. In some embodiments, the magnesium ions are added in a form of a magnesium salt. Non limiting examples of magnesium salts include: magnesium chloride, magnesium fluoride, magnesium sulfate, magnesium nitrate, magnesium acetate, magnesium carbonate, magnesium citrate, magnesium phosphate or hydrates thereof. Alternatively, the magnesium ions source may be a nanoparticle comprising magnesium ions. In some embodiment, the source of magnesium ions are capsules with antifouling properties which are spherical particles that can entrap and release different molecules. For a non-limiting example, the particles may be generated through a self-assembly of an antifouling peptide such as disclosed in Maity et al., Chemical communications (2014) 50, 11154-11157. In one embodiment, the source of magnesium ions are capsules that contain magnesium and can release magnesium ions.

In some embodiments, there is provided a method for inhibiting and/or reducing biofilm formation in a milk and/or a milk product, the method comprises the step of: adding magnesium ions to the milk or milk product to produce magnesium enriched milk and/or milk product, wherein the final concentration of magnesium ions in the magnesium enriched milk and/or milk product ranges from 7 mM to 30 mM, thereby reducing biofilm formation in said milk or said milk product.

In some embodiments, there is provided a method for improving efficiency of milk pasteurization, the method comprises the step of: adding magnesium ions to said milk or milk product to produce magnesium enriched milk and/or milk product, wherein the final concentration of magnesium ions in said magnesium enriched milk and/or milk product ranges from 7 mM to 30 mM, thereby improving susceptibility of bacteria to pasteurization. In some embodiments, the method further comprises a step of subjecting the magnesium enriched milk and/or milk product to pasteurization.

Biofilm

As used herein the term “biofilm” refers to any three-dimensional, matrix-encased microbial community displaying multicellular characteristics. Accordingly, as used herein, the term biofilm includes surface-associated biofilms as well as biofilms in suspension, such as flocs and granules. Biofilms may comprise a single microbial species or may be mixed species complexes, and may include bacteria, or other microorganisms.

In some embodiments, the biofilm comprises bacteria. In some embodiments, the bacteria are selected from: Gram positive bacteria and Gram negative bacteria. In some embodiments, the biofilm comprises bacteria. In some embodiments, the bacteria are Gram positive bacteria. In some embodiments, the bacteria are Gram negative bacteria. In some embodiments, the bacteria are spore forming bacteria. In some embodiments, the bacteria are thermophilic bacteria. The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are Gram-negative or Gram-positive.

In some embodiments, the bacteria are selected from the genera consisting of: bacillus, geobacillus, anoxybasillus, and pseudomona.

In some embodiments, the bacteria are selected from the bacteria strains: Bacillus cereus, Bacillus subtilis, Geobacillus stearothermophilus, Anoxybacillus flavithermus, and Pseudomonas aeruginosa.

Disinfectant

The present invention further relates to a disinfectant comprising an effective concentration of magnesium ions. A skilled artisan will appreciate that an effective concentration may depend on a specific use of the disinfectant. The present invention further relates to a disinfectant comprising magnesium ions in a concentration range of 50 mM to 500 mM. In some embodiments, the concentration of magnesium ions in the disinfectant ranges from 5 mM to 10 mM, 5 mM to 50 mM, 5 mM to 100 mM, 5 mM to 150 mM, 5 mM to 200 mM, 5 mM to 500 mM, 10 mM to 50 mM, 10 mM to 100 mM, 10 mM to 150 mM, 10 mM to 200 mM, 10 mM to 500 mM, 20 mM to 50 mM, 20 mM to 100 mM, 20 mM to 150 mM, 20 mM to 200 mM, 20 mM to 500 mM, 30 mM to 50 mM, 30 mM to 100 mM, 30 mM to 150 mM, 30 mM to 200 mM, 30 mM to 500 mM, 50 mM to 100 mM, 50 mM to 150 mM, 50 mM to 200 mM, 50 mM to 500 mM, 100 mM to 150 mM, 100 mM to 200 mM, 100 mM to 300 mM, 100 mM to 500 mM, or 100 mM to 1000 mM. Each possibility represents a separate embodiment of the present invention. The present invention further relates to a disinfectant comprising magnesium ions in a concentration of at least 5 mM, 10 mM, 15 mM, 20 mM, 25 mM, 30 mM, 35 mM, 40 mM, 45 mM, 50 mM, 55 mM, 60 mM, 65 mM, 70 mM, 80 mM, or 100 mM. In some embodiments, the disinfectant is an aqueous solution or a colloid solution. In one embodiment, the disinfectant is in a form of a foam or a spray. In one embodiment, the disinfectant is in a form of a cream.

In some embodiments, the disinfectant is for use in a method for treating, preventing, inhibiting, and/or reducing biofilm formation and/or reducing or breaking-down existing biofilms on a surface. In some embodiments, the disinfectant is for use in reducing an amount of bacterial biofilm formation on a surface. In another embodiment, the disinfectant is for use in cleaning machines in the food industry. In some embodiments, the biofilm is formed by bacteria. In some embodiments, populations of said bacteria may be treated by the disinfectant prior to, during, and/or after biofilm formation.

In some embodiments, there is provided a method for treating, preventing, inhibiting, and/or reducing biofilm formation and/or reducing or breaking-down existing biofilms on a surface, the method comprises the step of applying a composition comprising an effective concentration of magnesium ions onto said surface, thereby treating, preventing, inhibiting, and/or reducing biofilm formation and/or reducing or breaking-down existing biofilms on said surface.

In some embodiments, there is provided a method for treating, preventing, inhibiting, and/or reducing biofilm formation and/or reducing or breaking-down existing biofilms on a surface, the method comprises the steps of: providing a composition comprising an effective concentration of magnesium ions; and contacting said surface with said composition. In some embodiment the effective concentration of magnesium ions ranges from 20 mM to 500 mM. In some embodiment the effective concentration of magnesium ions is at least 20 mM.

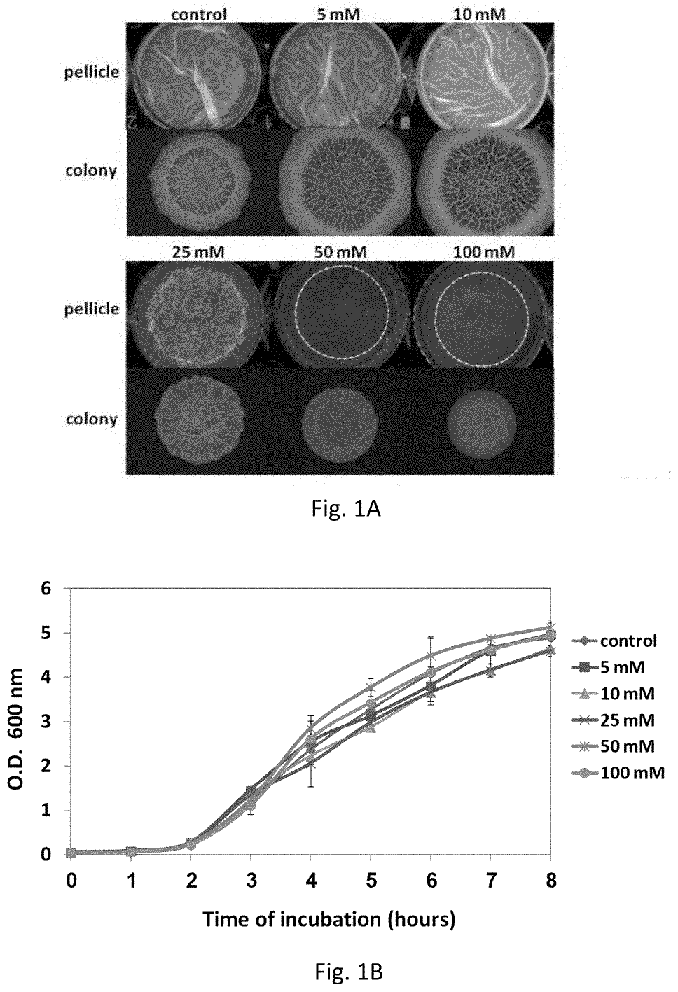

As exemplified in the example section below (see FIG. 1D), spreading the disinfectant comprising magnesium ions in a concentration of 20 mM or 50 mM onto a solid surface resulted in reduced biofilm formation by B. subtilis.

In some embodiments, the disinfectant is applied onto a surface. Any surface can be treated by the disinfectant. Examples of types of surfaces that may be treated by the disinfectant include, but are not limited to, food processing equipment surfaces such as tanks, conveyors, floors, drains, coolers, freezers, equipment surfaces, walls, valves, belts, pipes, joints, crevasses, combinations thereof, and the like. The surfaces can be metal, for example, aluminum, steel, stainless steel, chrome, titanium, iron, alloys thereof, and the like. The surfaces can also be plastic, for example, polyolefins (e.g., polyethylene, polypropylene, polystyrene, poly(meth)acrylate, acrylonitrile, butadiene, ABS, acrylonitrile butadiene, etc.), polyester (e.g., polyethylene terephthalate, etc.), and polyamide (e.g., nylon), combinations thereof, and the like. The surfaces may also be brick, tile, ceramic, porcelain, wood, vinyl, linoleum, or carpet, combinations thereof, and the like. The surfaces may also, in other aspects, be food, for example, beef, poultry, pork, vegetables, fruits, seafood, combinations thereof, and the like.

For disinfection and sterilization of hard surfaces, the disinfectant may be applied to the hard surface directly from a container in which the disinfectant solution is stored. For example, the disinfectant solution can be poured, sprayed or otherwise directly applied to the hard surface. The disinfectant solution can then be distributed over the hard surface using a suitable substrate such as, for example, cloth, fabric, or paper towel. Alternatively, the disinfectant may first be applied to a substrate such as cloth, fabric or paper towel. The wetted substrate can then be contacted with the hard surface. Alternatively, the disinfectant solution can be applied to hard surfaces by dispersing the solution into the air.

Kits

According to another aspect, the invention provides a kit comprising a composition of the invention together with packaging material.

In some embodiments, the invention provides an article comprising any of the compositions of the invention. In some embodiments, the article is selected from the group consisting of a food package, a milk production and/or processing device.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); “Bacteriophage Methods and Protocols”, Volume 1: Isolation, Characterization, and Interactions, all of which are incorporated by reference. Other general references are provided throughout this document.

Various embodiments and aspects of the present invention as delineated hereinabove and as claimed in the claims section below find experimental support in the following examples. Reference is now made to the following examples, which together with the above descriptions illustrate some embodiments of the invention in a non-limiting fashion.

Materials and Methods

Bacteria Strains and Growth Media

The Bacillus subtilis wild strain NCIB3610 and Bacillus cereus ATCC 10987 stain, were used in this study. For fluorescent microscopy, a strain (YC161 with Pspank-gfp) that produced GFP constitutively.

For routine growth, all strains were propagated in Lysogeny broth (LB; 10 g of tryptone, 5 g of yeast extract and 5 g of NaCl per liter) or on solid LB medium supplemented with 1.5% agar. For biofilm generation, bacteria were grown to stationary phase in LB medium at 37° C. in shaking culture to around 1×10⁸ CFU per ml. Biofilms were generated at 30° C. in the biofilm promoting medium LBGM (LB+1% (v/v) glycerol+0.1 mM MnSO4). To test the effect of magnesium, sodium or calcium ions on biofilm formation, different concentrations of either MgCl2 (Merck KGaA), NaCl (BIO LAB LTD) or CaCl2 (Merck KGaA) were added directly into the LBGM medium. For colony type biofilm formation, 3 μl of the cells (around 3×10⁵ CFU) was spotted onto LBGM medium solidified with 1.5% agar as described previously. Plates were incubated at 30° C. for 72 h prior to analysis. For pellicle formation, 5 μl of the cells (around 5×10⁵ CFU) was mixed within 4 ml of LBGM broth in 12-well plates (Costar). Plates were incubated at 30° C. for 24 h. Images were taken using a Zeiss Stemi 2000-C microscope with an axiocam ERc 5s camera.

For experiments performed with B. cereus, bacteria were grown to stationary phase in LB medium at 37° C. in shaking culture to around 5×10⁷ CFU per ml. For pellicle formation, 5 μl of the cells (around 2.5×10⁵ CFU) was mixed within 4 ml of LBGM broth in glass tubes in the presence or absence of different concentration of MgCl2. The glass tubes were incubated at 30° C. for 24 hours.

β-Galactosidase Activity

To analyze the effect of magnesium ions on matrix gene expression a transcriptional fusions of the promoters for eps and tapA to the gene encoding β galactosidase were used. Samples of generated pellicles as described above were collected and resuspended in phosphate-buffered saline (PBS) buffer. Typical long bundled chains of cells in the biofilm colony were disrupted using mild sonication as described previously. Optical density of the cell samples was normalized using OD600. One milliliter of cell suspensions was collected and assayed for (3-Galactosidase activity as described previously.

Growth Curve Analysis

Initially, the cells were grown in shaking cultures over night at 23° C./150 rpm in LB to around 2×10⁹ CFU per ml. On the next morning, the cultures were diluted 1:100 (to around 2×10⁷ CFU) into LBGM with or without addition of different concentration of MgCl2 and incubated at 37° C. at 150 rpm. The absorbance of the cultures at 600 nm was measured periodically for each culture for 9 hours. Each condition had 3 replicates, and the growth curve experiments were repeated twice. Representative results are shown.

Fluorescent Microscopy Analysis

For fluorescent microscopy, the strain YC161 that produced GFP constitutively, was used. The strain was first grown in shaking culture for 5 h 37° C./150 rpm in LB to around 1×10⁸ CFU per ml. Next, 5 μl (around 5×10⁵ CFU) of suspension from the generated culture was introduced into 4 ml of LBGM medium and incubated at 30° C. for 24 h statically. Afterwards, one milliliter of suspension from each sample was collected, mildly sonicated (10 sec/20% Amp/5) and centrifuged at 5000 rpm for 2 min. Next, the supernatant was removed and the pellet was re-suspended by pipetting. For microscopic observation, 34, from the samples were transferred onto a glass slide and visualized in a transmitted light microscope using Nomarski differential interference contrast (DIC), at ×40 magnification. A confocal laser scanning microscope was used to visualize GFP expression of strain YC161 using an Olympus IX81 confocal laser scanning microscope (Japan) equipped with 488 nm argon-ion and 543 nm helium neon lasers. For experiments performed with B. cereus, the cells were stained with CYTO 9 from the FilmTracer™ LIVE/DEAD Biofilm Viability Kit (Molecular Probes, OR) following instructions of the manufacturer. Fluorescence emission of the stained samples was determined using an Olympus IX81 confocal laser scanning microscope (Japan) equipped with 488 nm argon-ion and 543 nm helium neon lasers.

Statistical Analysis

Statistical analysis was performed using T-test to compare the control and tested samples. Statistical significant was determined at P<0.05.

### Example 1

### Magnesium Ions Inhibit Biofilm Formation

The effect of different concentrations of Mg′ ions on biofilm formation. Results demonstrated that Mg′ ions inhibited notably pellicle formation by B. subtilis in a concentration dependent manner (FIG. 1A). The inhibitory effect of Mg′ ions was not restricted to MgCl₂ compound since other magnesium salts, such as MgSO₄ have also inhibited the pellicle formation. This indicates that the inhibitory effect of magnesium salts is attributed to Mg′ ions. Moreover, colony type biofilm formation was also inhibited significantly in the presence of high concentrations of Mg′ ions (FIG. 1A).

The effect of different concentrations of Mg′ ions on bacterial growth was also examined. Results demonstrated that the presence of Mg′ ions did not affect bacterial growth at the tested concentrations (FIG. 1B).

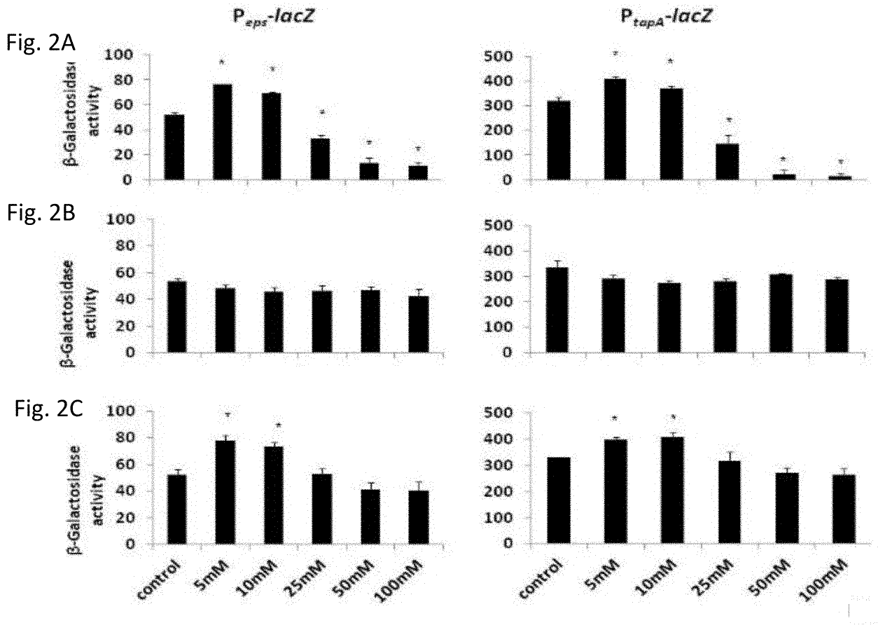

Next, the effect of magnesium ions was visualized microscopically by testing bundling phenotype of fluorescently tagged B. subtilis cells (YC161 with Pspank-gfp), that produce GFP constitutively. As demonstrated in FIG. 3, in the presence of 25 mM MgCl₂ and higher concentrations there is significant reduction in bundling ability of B. subtilis cells. This result further confirms the potential of Mg²⁺ ions to inhibit biofilm formation by B. subtilis.

Further, the effect of NaCl and CaCl₂ on biofilm formation by B. subtilis was examined. Notably, none of those compounds could inhibit the biofilm formation in the same manner as MgCl₂ (results not shown).

Additional experiments were conducted in order to evaluate the effect of magnesium ions on colony type biofilm formation by Bacillus subtilis on a solid surface. First a starter culture was prepared by growing bacteria from the strain Bacillus subtilis NCIB 3610 in an LB (Lysogeny broth) medium at 37° C., 150 rpm for 5 hours. Next, solutions having different concentrations of MgCl₂, namely, 0 mM (control), 5 mM, 20 mM, or 50 mM MgCl₂, were spread onto a different surface of a solid biofilm promoting medium (LBGM solidified with 1.5% agar). Following these steps, 3 μl of the starter culture were spotted onto each of the solid biofilm promoting mediums. The samples were incubated at 30° C. for 72 hours prior to analysis. Images were taken using a Zeiss Stemi 2000-C microscope with an axiocam ERc 5s camera.

As shown in FIG. 1D, pre-treating a surface of solid biofilm promoting medium (LBGM solidified with 1.5% agar) by spreading a solution of 20 mM MgCl₂ exhibits inhibitory effect on biofilm formation by B. subtilis onto the surface. The inhibitory effect is more significant when using a solution of 20 mM MgCl₂. These results suggest that a magnesium solution may be applied onto solid surfaces to reduce, inhibit, and/or prevent biofilm formation.

Further, the effect of magnesium ions on biofilm formation by B. subtilis within orange juice enriched medium, was examined. For this purpose, biofilm formation of by B. subtilis within orange juice enriched medium (LB+ orange juice) was compared to that of orange juice enriched medium supplemented with 50 Mm MgCl₂ (LB+ orange juice+50 Mm MgCl₂) and orange juice enriched medium supplemented with 80 Mm MgCl₂ (LB+ orange juice+80 Mm MgCl₂). The results demonstrate that MgCl2 inhibits colony type biofilm formation at 50 mM and higher (FIG. 1E).

### Example 2

### The Effect of Mg²⁺, Ca²⁺, and Na⁺ Ions on Transcription of the Operons Responsible for the Matrix Production

A skilled artisan will appreciate that biofilm formation is, at least partially, depended on the synthesis of extracellular matrix. The production of the extracellular matrix in B. subtilis is specified by two major operons: the epsA-O and tapA operons. The epsA-O operon is responsible for the production of the exopolysaccharides whereas the tapA operon is responsible for the production of amyloid-like fibers.

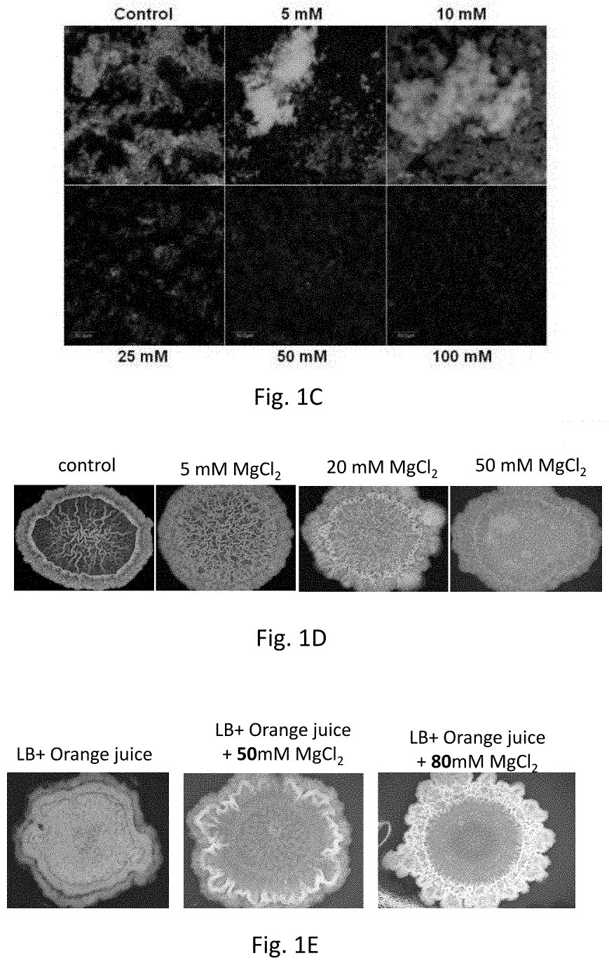

The effect of Mg²⁺ ions on matrix gene expression was examined by using transcriptional fusions of the promoters for epsA-O and tapA to the gene encoding β galactosidase. The expression of the matrix operons was notably reduced in response to the addition of Mg²⁺ ions (FIG. 2A). The reduction in eps expression was relatively small (around 4-fold) but significant, while tapA expression was decreased almost 14.5-fold at elevated concentrations of Mg²⁺ ions (FIG. 2A). This result suggests that addition of Mg′ ions down regulates expression of the extracellular matrix genes in B. subtilis. Notably, in the presence of similar concentrations of either NaCl (FIG. 2B) or CaCl₂ (FIG. 2C), the expressions of the eps and tapA operons were not downregulated in a similar manner.

### Example 3

### Magnesium Ions Inhibit Biofilm Formation within Milk

The effect of magnesium ions on biofilm formation within cow's milk was examined. A skilled artisan will appreciate that magnesium concentration within cow's milk typically ranges between 4-6 mM. In order to examine the effect of magnesium ions, bacteria were grown in cow's milk cow's milk supplemented with MgCl₂ to obtain cow's milk having a concentration of magnesium ions increase of 1 mM, 3 mM or 5 mM. Results demonstrated that Mg′ ions inhibit biofilm formation by B. subtilis within milk. The inhibitory effect of Mg2+ ions is notable even when the magnesium concentration is increased by 1 mM compared to control. Further, at 5 mM increase of MgCl₂ concentration the biofilm bundles are almost totally diminished (FIG. 3A). Notably, similarly to the results of FIG. 1B, bacterial growth was not significantly affected by increase of Mg′ ions concentration within milk (FIG. 3B).

### Example 4

### Increase in Magnesium Ions Concentration Downregulates the Expression of tapA Operon

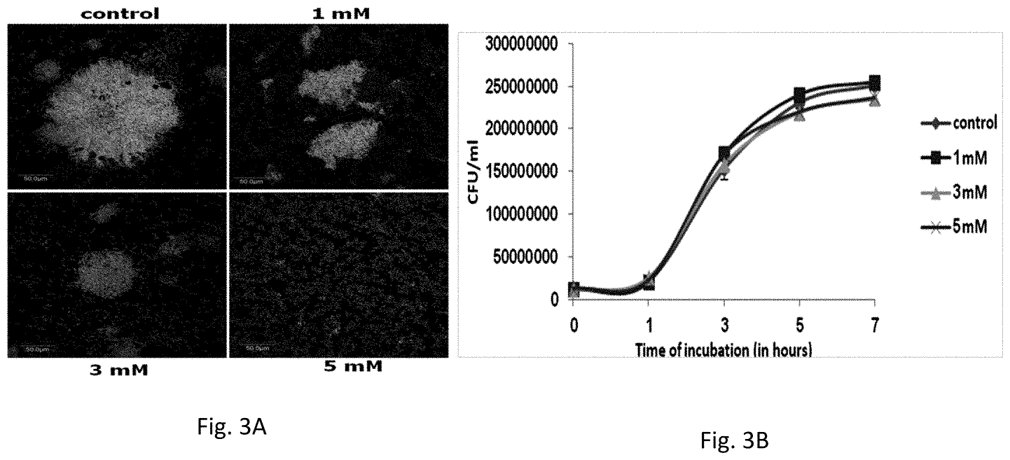

The effect of increase in concentration of Mg′ within milk on the expression of tapA operon, which is one of the major operon for matrix production, was examined. For this purpose, B. subtilis were grown within cow's milk and cow's milk with an increase of 1 mM, 3 mM or 5 mM in concentration of magnesium ions. Further, transcriptional fusions of tapA promoter to the gene encoding cyan fluorescent protein (CFP) were utilized. Results demonstrated that the expression of the tapA operon is reduced drastically in response to increase in Mg²⁺ ions within milk, particularly when 5 mM MgCl2 concentration is added (FIG. 4).

### Example 5

### Increase in Magnesium Ions Concentration Increases Bacteria Susceptibility to Heat Stress

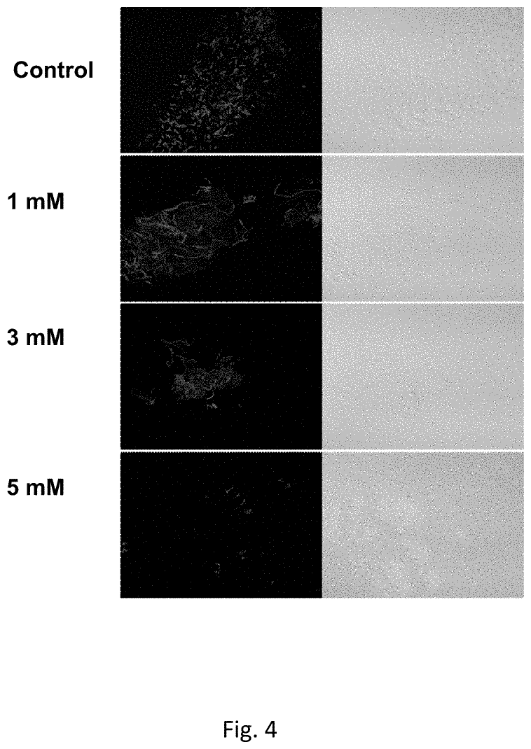

The effect of increase in the concentration of magnesium ions was examined. B. subtilis were grown within milk or milk supplemented with additional 3 mM or 5 mM MgCl₂ or 3 mM or 5 mM CaCl₂. The survival rate of bacteria following pasteurization was examined. Results demonstrated that increase in the concentration of Mg²⁺ ions increase the susceptibility of bacteria to heat pasteurization (FIG. 5).

### Example 6

### Increase in Magnesium Ions Concentration Improves Milk Clotting Parameters

In order to examine milk clotting parameters, milk samples obtained from the dairy farm of the Agricultural Research Organization (ARO; Bet Dagan, Israel). Milk samples were compared to milk samples supplemented with either MgCl₂ or CaCl₂ resulting in 3 mM or 5 mM increase in magnesium ions or calcium ions, respectively. Milk-clotting parameters such as rennet clotting time (RCT; min) and curd firmness (CF; V) after 90 min (CF-90) which are measured with an Optigraph instrument (Ysebaert, Frepillon, France) as described by Leitner et al. (2011). Results are presented in a graph. The point in which the curve is split into two curves, represents the rennet Clotting Time (RCT), which is the time between the addition of the clotting enzyme and the beginning of the coagulation process. The curd firmness is derived from the distance of the two curves in the graph 30 min after the addition of the clotting enzyme, or 90 minutes after the addition of the clotting enzyme.

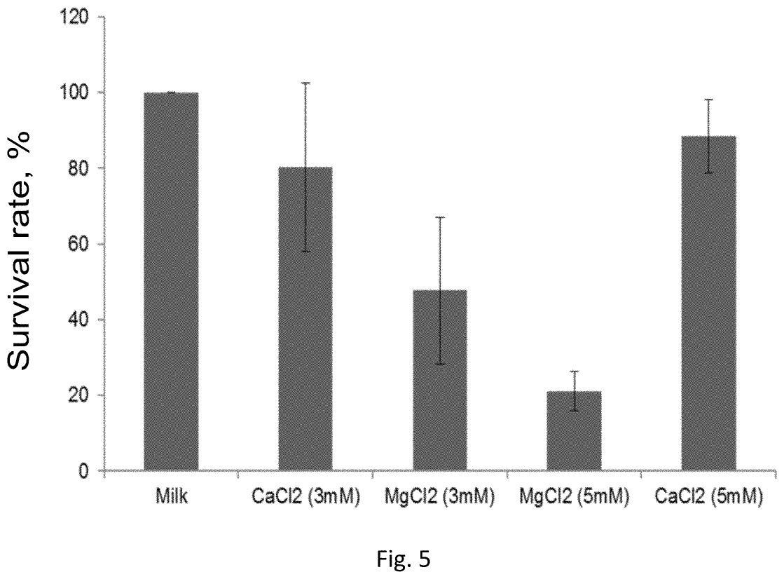

Results demonstrate that the RCT is significantly lower in magnesium enriched milk having a 5 mM increase in concentration of magnesium ions compared to control un-supplemented milk; whereas, the CF-90 is notably higher in the sample supplemented by Mg′ ions (FIG. 6).

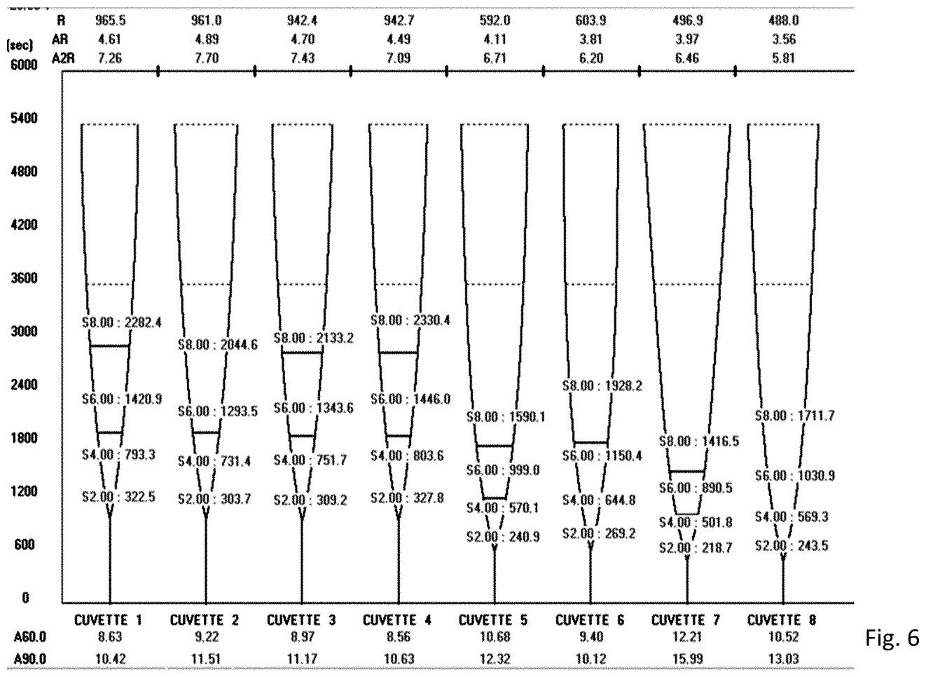

As further shown in FIG. 7, the resulting magnesium enriched cheeses demonstrate increased curd firmness, suggesting that the curdling process improves in the presence of increase of either 3 mM or 5 mM in the concentration of magnesium ions.

RCT and CF measurements of control un-supplemented milk samples and milk samples supplemented with 3 mM or 5 mM MgCl₂ are summarized in table 1.

TABLE 1
RCT and CF

| | RCT (minutes) | | | Curd firmness (volts) | | |
|---|---|---|---|---|---|---|
| | Control | 3 mM Mg | 5 mM Mg | Control | 3 mM Mg | 5 mM Mg |
| | 19.46 | 11.13 | 7.25 | 7.96 | 10.83 | 13.36 |
| | 18.49 | 11.09 | 7.53 | 8.88 | 10.37 | 11.49 |
| | 26.46 | 10.63 | 9.97 | 6.69 | 13.03 | 12.75 |
| | 24.29 | 10.49 | 9.87 | 8.29 | 12.90 | 13.35 |
| | 24.98 | 12.84 | 9.47 | 7.02 | 12.04 | 13.23 |
| | 24.51 | 12.87 | 9.59 | 7.94 | 12.42 | 12.73 |
| | 18.44 | 13.31 | 8.49 | 8.73 | 11.18 | 15.07 |
| | 17.64 | 13.04 | 8.63 | 10.26 | 11.87 | 17.77 |
| | 24.93 | 12.96 | 10.31 | 7.23 | 11.76 | 13.01 |
| | 25.00 | 12.89 | 10.31 | 7.57 | 12.38 | 13.21 |
| | 27.54 | | 10.20 | 7.40 | | 13.57 |
| | 26.22 | | 10.40 | 7.15 | | 12.71 |
| | 27.18 | | 9.97 | 8.42 | | 14.45 |
| | 25.56 | | 9.93 | 6.90 | | 13.19 |
| Average | 23.62 | 12.12 | 9.42 | 7.89 | 11.88 | 13.56 |

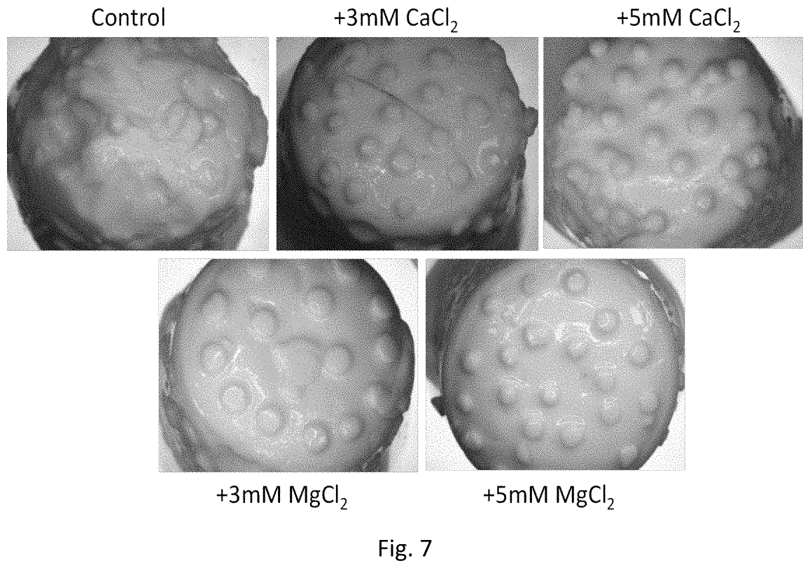

In further experiments milk samples were compared to milk samples supplemented with MgCl₂ resulting in 1 mM, 3 mM, 5 mM, 7 mM, 10 Mm, 15 mM or 20 mM increase in magnesium ions. As demonstrated in FIG. 8A addition of 1 mM, 3 mM, 5 mM, 7 mM, 10 mM, 15 mM, and 20 mM MgCl₂ results in about 32%, 51%, 62%, 70%, 84%, 73%, and 74% decrease in RCT, respectively. As demonstrated in FIG. 8B addition of 1 mM, 3 mM, 5 mM, 7 mM, 10 mM, 15 mM, and 20 mM MgCl₂ results in about 37%, 54%, 69%, 82%, 84%, 90%, and 72% increase in curd firmness, respectively. Results indicated that as the concentration of magnesium ions increases the RCT decreases (see curding beginning time FIG. 8A) and curd firmness increases (FIG. 8B). Notably, these results further indicate that addition of 20 mM does not results in increased curd firmness compared to addition of 15 mM MgCl₂ (FIGS. 8A and 8B).

### Example 7

### Increase in Magnesium Ions Improves Incorporation of Proteins into Soft Cheese

The effect of increased concentration of Mg²⁺ ions on the incorporation of milk proteins into the soft cheeses was examined.

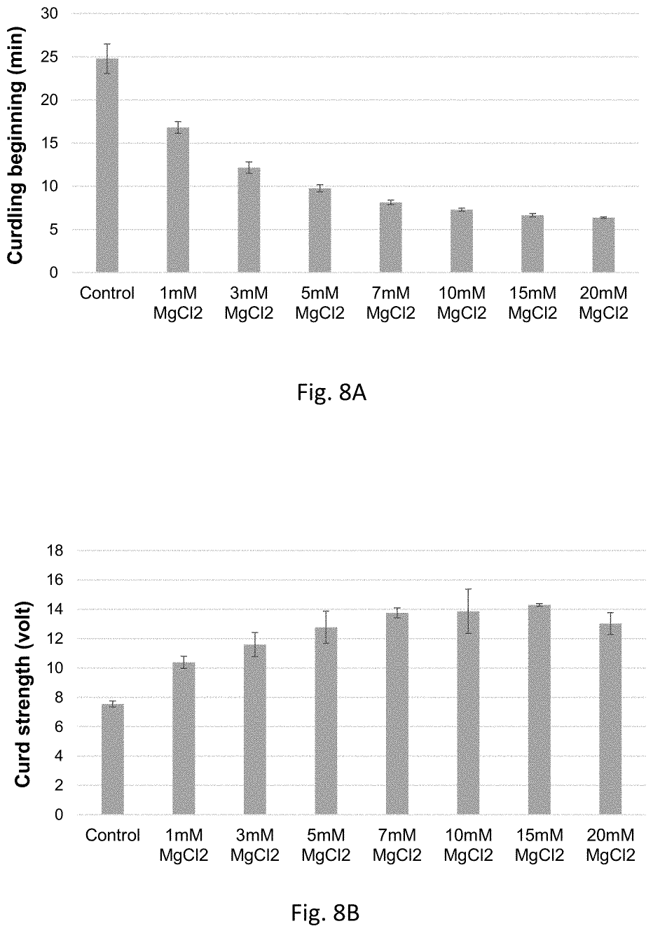

Milk was obtained from the dairy farm of ARO and pasteurized in water bath at 63° C. for 30 minutes. The cheeses samples were prepared from 50 milliliters (ml) milk with or without addition of MgCl₂, resulting in 5 mM increase concentration in the concentration of magnesium ions. 2.5 ml of the enzyme “Renin” was added to each sample. The samples were incubated in water bath at 30° C. for 1 hour. Next, the cheese samples were cut and put into water bath at 40° C. for 30 minutes in order to drain the whey. Afterwards, the cheese samples were transferred into perforated tubes and kept at 4° C. for 24 hours to remove the whey. Subsequently, the levels of protein were determined using Kjeldahl method. Results demonstrated that cheese prepared from magnesium enriched milk (supplemented with additional 5 mM MgCl₂) exhibits increase in the incorporated protein, compared to a cheese prepared from un-supplemented milk (FIG. 9).

### Example 8

### Bacillus subtilis Bacteria in the Presence of High Concentrations of MgCl₂ Cause Protein Precipitation in Milk

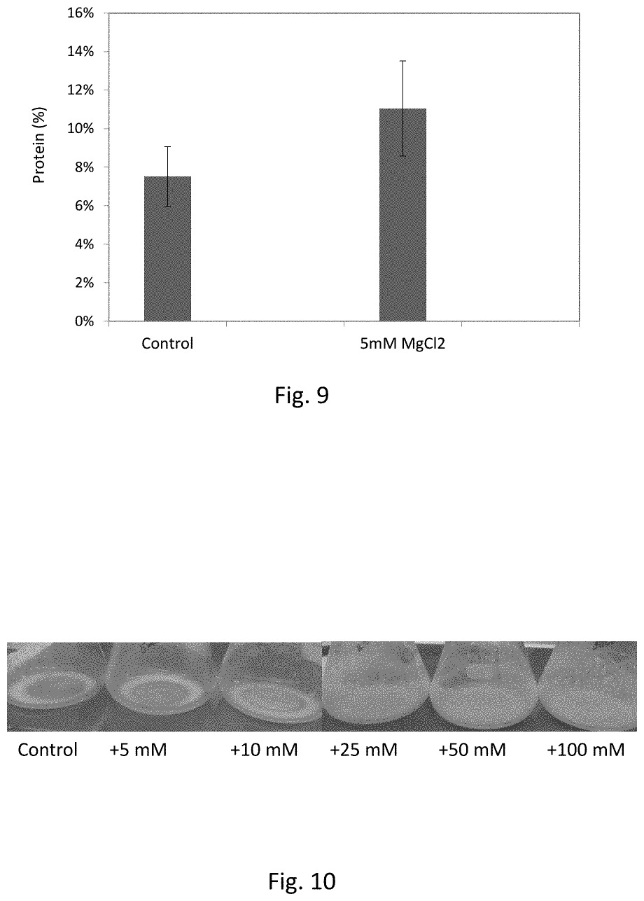

Overnight cultures of Bacillus subtilis were diluted (a 1:100 dilution) into milk or milk supplemented with different concentration of MgCl₂. Cultures were then incubated for 5 hours at 37° C. and 50 rpm. Results demonstrate that when a concentration of 25 mM MgCl₂ or more was added to milk (typically having a base concentration range of 4-6 mM of magnesium ions), Bacillus subtilis bacteria caused protein precipitation in the milk (FIG. 10).

### Example 9

### MgCl₂ Supplementation does not Affect the pH of the Milk

Overnight cultures of Bacillus subtilis were diluted (a 1:100 dilution) into milk or milk supplemented with 50 mM or 100 mM of MgCl₂. Cultures were incubated for 5 hours at 37° C. and 50 rpm and pH was measured every hour using pH strips. Results demonstrate similar pH measurements in the milk supplemented with either 50 mM or 100 mM of MgCl₂ and the un-supplemented milk. These results suggest that MgCl₂ supplementation does not affect the pH of the milk.

1. A method of milk processing, the method comprising:
adding an effective amount of magnesium ions source to milk, thereby producing magnesium enriched milk;
wherein the effective amount is sufficient for increasing magnesium concentration in said magnesium enriched milk in a range from 1 mM to 20 mM.

2. The method of claim 1, wherein said milk is a pasteurized milk.

3. The method of claim 1, further comprising a step of clotting said magnesium enriched milk, thereby obtaining a milk product.

4. The method of claim 3, wherein said clotting comprises enzymatic coagulation, or acidic coagulation.

5. The method of claim 3, wherein said milk product is a fermented milk product.

6. The method of claim 3, wherein said method is for any one of: (i) increasing protein incorporation into a curd, and (ii) increasing milk clotting, compared to a non-enriched milk obtained from the same mammal.

7. The method of claim 1, wherein said method is for reducing rennet clotting time (RCT) of said magnesium enriched milk, compared to a non-enriched milk obtained from the same mammal.

8. The method of claim 1, wherein said method is for reducing and/or inhibiting biofilm formation in said magnesium enriched milk, compared to a non-enriched milk obtained from the same mammal.

9. The method of claim 1, wherein said increasing magnesium concentration is in a range from 3 mM to 15 mM.

10. The method of claim 1, wherein said magnesium ions source is a magnesium salt.

11. A milk product obtained from a magnesium enriched milk processed from claim 1,
wherein:
the milk product is characterized by at least one of: (i) increased protein levels, compared to a milk product processed from non-magnesium-enriched milk obtained from the same mammal; and (ii) increased curd firmness (CF, min), compared to a milk product processed from non-magnesium-enriched milk obtained from the same mammal; and
wherein a concentration of magnesium ions within the magnesium enriched milk is between 12 millimol per liter (mM) and 20 mM.

12. The milk product of claim 11, wherein said milk product is a fermented milk product.

13. The milk product of claim 12, wherein said fermented milk product is selected from the group consisting of yoghurt, kefir, curd cheese, curd, buttermilk, fresh cheese and semi-solid cheese or any combination thereof.
